(19) United States
(12) Patent Application Publication
(10) Pub. No.: US 2026/0306367 A1
(43) Pub. Date: Oct. 8, 2026

(54) Solid Composition Comprising Natural Dyes and Makeup Process Using Same

(71) Applicant: L'OREAL, PARIS (FR)

(72) Inventors: LUDOVIC THEVENET, CHEVILLY LARUE (FR); LAURE LE CHAUX, CHEVILLY LARUE (FR)

(73) Assignee: L'OREAL, Paris (FR)

(21) Appl. No.: 19/481,319

(22) PCT Filed: Apr. 30, 2024
(86) PCT No.: PCT/EP2024/061854

§ 371 Date: Nov. 4, 2025

(30) Foreign Application Priority Data
May 5, 2023 (FR) 2304500

(51) Int. Cl. A61K 8/02; A61K 8/29; A61K 8/31; A61K 8/37; A61K 8/67; A61K 8/73; A61K 8/85; A61K 8/892; A61K 8/92; A61Q 1/06

(52) U.S. Cl. CPC A61K 8/0229; A61K 8/29; A61K 8/31; A61K 8/375; A61K 8/671; A61K 8/678; A61K 8/731; A61K 8/85; A61K 8/892; A61K 8/922; A61K 8/927; A61Q 1/06; A61K 2800/43; A61K 2800/48

(57) ABSTRACT

The present invention relates to a solid cosmetic composition for making up and/or caring for keratin materials such as the skin and/or the lips, comprising: (A) at least one natural dye chosen from anthocyanins, anthocyanidins, proanthocyanidins, and mixtures thereof; (B) at least one first compound that is solid at 25° C., chosen from polyesters obtained at least from a monounsaturated or polyunsaturated fatty acid dimer; the fatty acid comprising from 16 to 22 carbon atoms; (C) at least one volatile or non-volatile first oil, chosen from apolar hydrocarbon oils; silicone oils; liquid polyesters obtained from a monounsaturated or polyunsaturated fatty acid dimer, the fatty acid comprising from 16 to 22 carbon atoms; ester, ether or carbonate hydrocarbon oils, these oils being other than the abovementioned liquid polyesters and being free of free hydroxyl groups; plant oils with the exception of castor oil and of lesquerella oil; and mixtures thereof. It also relates to a makeup process consisting in applying such a composition.

The present invention relates to a solid composition for making up and/or caring for the skin or the lips, comprising at least one natural dye or dye of natural origin and at least one solid compound obtained from a monounsaturated or polyunsaturated fatty acid dimer.

Makeup compositions usually comprise synthetic coloured species, which are soluble or dispersed in the medium of the composition, or alternatively coloured species of natural origin dispersed in the medium of the composition, for instance pearlescent agents. The situation is, however, more complex if it is desired to use natural dyestuffs, in particular such as anthocyans, as sole dyestuffs, or in a significant amount relative to the other coloured substances. The reason for this is that the colour of these substances is usually pH-sensitive, which may entail, if these dyes are used in sufficient amount, an undesirable variation in the colour of the composition when it is applied to a support such as the lips, for example. Moreover, in the more specific case of natural dyes of the anthocyanin type, since most of these dyes are soluble in water, using them in a buffered aqueous phase does not represent an appropriate solution either. The appearance of a very dark precipitate, which can no longer be exploited, is in fact then observed.

Consequently, one of the objectives of the present invention is to propose solid compositions for making up and/or caring for human keratin materials, more particularly the skin and the lips, comprising at least one natural dye chosen from anthocyans, the colour of which does not vary significantly, even when these dyes are employed as sole dyestuffs or as main dyestuffs.

Thus, a subject of the present invention is a solid composition for making up and/or caring for keratin materials, such as the skin and/or the lips, and preferably the lips, comprising:
(A) at least one natural dye chosen from anthocyanins, anthocyanidins, proanthocyanidins, and mixtures thereof;
(B) at least one first compound that is solid at 25° C., chosen from polyesters obtained at least from a monounsaturated or polyunsaturated fatty acid dimer; the fatty acid comprising from 16 to 22 carbon atoms;
(C) at least one volatile or non-volatile first oil, chosen from apolar hydrocarbon oils; silicone oils; liquid polyesters obtained from a monounsaturated or polyunsaturated fatty acid dimer, the fatty acid comprising from 16 to 22 carbon atoms; ester hydrocarbon oils other than the aforementioned liquid polyesters, ether hydrocarbon oils or carbonate hydrocarbon oils, these oils being free of free hydroxyl groups; plant oils with the exception of castor oil and of lesquerella oil; and mixtures thereof.

A subject of the present invention is also a process for making up keratin materials, such as the skin and/or the lips, and in particular the lips, consisting in applying the composition as described above.

### Protocol for Measuring the Colour Variation

The composition is applied to the bare lips of six subjects so as to cover the lips uniformly (more particularly 2 to 3 back and forth movements on the upper lip, 2 to 3 back and forth movements on the lower lip; if the composition is not in the form of a wand, it is applied by finger or by means of an applicator; the upper lip is made up without intermediate product refill, likewise for making up the lower lip).

Measurement of the colour of the made-up lips is then performed in a Chromalys Samba-Shiva chromasphere, according to the method described in the patent FR 2829344 (SAMBA polarimetric camera; definition 410×410 pixels)
immediately after application, i.e. 3 to 5 minutes after application,
1 hour after application, without eating or drinking between the application and the measurement.

The coordinates L*, a*, b*, of the colour are measured in the CIELab space 76 (illuminant D65).

To evaluate the variation in colouration, the ΔE₂₀₀₀ is calculated according to the ISO 11664-6:2014 standard (specification of the method of calculation of the colour differences according to the formula CIEDE2000) for each subject and the mean of the six values is calculated.

It is considered that the composition does not change colour when the mean value of ΔE₂₀₀₀ directly after application and 1 hour thereafter is less than or equal to 2, more particularly less than or equal to 1.5 and even more particularly less than or equal to 1.

The composition according to the invention is in a solid form. The term “solid composition” denotes a composition which does not flow under its own weight at 25° C. and atmospheric pressure, after one hour. The composition can thus be in a solid form in a jar or pallet or in the form of a wand.

### Protocols for Measuring the Hardness of a Wand Composition

The composition in wand form is stored at 20° C. for 24 hours before measuring the hardness.

The measurement is performed at 20° C. and consists in transversely cutting a wand of product, which is preferably a circular cylinder, by means of a rigid tungsten wire 250 μm in diameter, by moving the wire relative to the stick at a speed of 100 mm/minute.

The hardness of the samples of compositions of the invention, expressed in Nm-1, is measured using a DFGS2 tensile testing device sold by Indelco-Chatillon.

The measurement is repeated three times and then averaged. The mean of the three values read using the tensile testing machine mentioned above, noted Y, is given in grams. This mean is converted into newtons and then divided by L which represents the longest dimension through which the wire passes. In the case of a cylindrical wand, L is equal to the diameter (in metres). The hardness is converted into Nm⁻¹ by the following equation (Y×10⁻³× 9.8)/L.

According to this measurement method, the composition according to the invention, when it is in solid form, advantageously has a hardness at 20° C. and at atmospheric pressure of between 80 and 200 Nm⁻¹, and preferentially between 100 and 200 Nm⁻¹, even more particularly between 100 and 150 Nm⁻¹, or even 130 Nm⁻¹.

### Natural Dyestuffs or Dyestuffs of Natural Origin (A)

The composition according to the invention comprises at least one dyestuff chosen from particular natural compounds.

A “natural compound” is understood to mean a compound obtained directly from the earth or the soil, or from plants or animals, via, where appropriate, one or more physical processes, such as for example milling, refining, distillation, purification or filtration, or else obtained from a biotechnological process, in particular obtained from microbiological or cell cultures, for example from fungi or from bacteria.

A compound “of natural origin” is understood to mean a natural compound that has undergone one or more supplementary chemical or industrial treatments, giving rise to modifications that do not affect the essential qualities of this compound and/or a compound predominantly comprising natural constituents that may or may not have undergone transformations as indicated above. As non-limiting examples of supplementary chemical or industrial treatments giving rise to modifications that do not affect the essential qualities of a natural compound, mention may be made of those permitted by the regulatory bodies such as COSMOS (Reference system for cosmetic, biological and ecological products, version 3.1 of 1 Jun. 2020), or possibly defined by the AFNOR standard 16-128.

The term “dye” and more generally “water-soluble compound” means a dye or a compound which has a solubility in water, measured at 25° C., at least equal to 0.01 g/l (production of a macroscopically isotropic, transparent, coloured or colourless solution).

As has been indicated previously, the composition according to the present invention thus comprises at least one natural dyestuff or dyestuff of natural origin chosen from anthocyanins, anthocyanidins, proanthocyanins, proanthocyanidins, and also mixtures thereof, and preferably chosen from anthocyanins, anthocyanidins, and mixtures thereof.

As regards the proanthocyanidins, the proanthocyanidins A1, A2, B1, B2, B3 and C1 may in particular be suitable for performing the invention.

As regards the anthocyanins, they are substituted glycoside salts and acyl glycosides of 2-phenylbenzopyrylium.

Anthocyanidins are aglycone anthocyanin compounds.

The basic structure of anthocyanidins is constituted of a chroman nucleus (C₆-ring A and C₃-ring C) bearing a second aromatic ring (C₆-ring B) in position 2, as represented in the formula below:

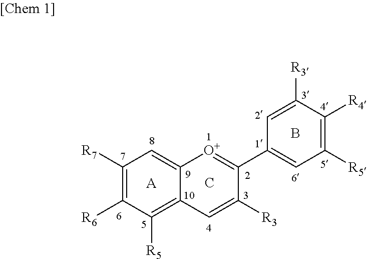

| | | | | | | | |
|---|---|---|---|---|---|---|---|
| 1 | Pelargonium (Pg) | R₃═OH; | R₅═OH; | R₆═H; | R₇═OH; | R_{3′}═H; | R_{4′}═OH; |
| | R_{5′}═H | | | | | | |
| 2 | Cyanidin (Cy) | R₃═OH; | R₅═OH; | R₆═H; | R₇═OH; | R_{3′}═OH; | R_{4′}═OH; |
| | R_{5′}═H | | | | | | |
| 3 | Delphinidin (Dp) | R₃═OH; | R₅═OH; | R₆═H; | R₇═OH; | R_{3′}═OH; | R_{4′}═OH; |
| | R_{5′}═OH | | | | | | |
| 4 | Peonidin (Pn) | R₃═OH; | R₅═OH; | R₆═H; | R₇═OH; | R_{3′}═OMe; | R_{4′}═OH; |
| | R_{5′}═H | | | | | | |
| 5 | Petunidin (Pt) | R₃═OH; | R₅═OH; | R₆═H; | R₇═OH; | R_{3′}═OMe; | R_{4′}═OH; |
| | R_{5′}═OH | | | | | | |
| 6 | Malvidin (Mv) | R₃═OH; | R₅═OH; | R₆═H; | R₇═OH; | R_{3′}═OMe; | R_{4′}═OH; |
| | R_{5′}═OMe | | | | | | |

The various anthocyanidins differ in the number and position of the hydroxyl and/or methyl ether groups attached to their position 3, 5, 6, 7, 3′, 4′ and/or 5′.

Over thirty one monomeric anthocyanidins have been identified. However, 90% of the naturally existing anthocyanins are represented by six structures (30% cyanidins (2), 22% delphinidins (3), 18% pelargonidins (1) and the final 20% made up of peonidins (4), malvidins (6) and petunidins (5)).

These six anthocyanidins are the most well-known anthocyanidins.

The colour of the anthocyanidins differs with the number of hydroxyl groups attached to the molecules and more particularly those present in ring B.

The colour of the molecule changes from orange to purple with the number of hydroxyl units present in its structure.

Glycosylation of the anthocyanidins has the effect of stimulating the red colour thereof, while the presence of aromatic or aliphatic acyl units has an essential effect on the stability and solubility thereof.

These dyes may notably be characterized by the following CAS numbers: 134-01-1, 134-04-3, 528-53-0, 528-28-5, 643-84-5, 1429-30-7, 11029-12-2, etc., and defined by the “CTFA—International Color Handbook”, Third Edition, CTFA, pages 856-857.

These dyes may be found in many plants, such as for instance chokeberry, blueberry, cranberry, purple or black carrot, blackcurrant, cherry, red cabbage, poppy, strawberry, geranium, pomegranate, hibiscus, purple corn, bilberry, purple sweet potato, red radish, black grape, sorghum and elderberry.

Preferably, the dyes are chosen from those originating from radish (INCI name: Raphanus sativus root extract), from purple sweet potato (INCI name: Ipomoea batatas root extract).

The dyes used in the context of the present invention may be in pulverulent form, in the form of an extract, in solution form, in emulsion form or in dispersion form.

Among the commercial products that are suitable for performing the invention, mention may be made of the products CD Radish Red Powder C (Omya), Natpure Xfine Red Radish or Natpure Xfine Purple Sweet Potato (Sensient Cosmetic Technologies) and Microzest 25 Carrot Pink (Lessonia), New Red 1805 (Naturex).

The dyes described in patent application WO 2017/162599 may also be suitable for use.

The content of natural dyestuff(s) or dyestuff(s) of natural origin, expressed as dyestuff active material, is more particularly between 0.5% and 20% by weight, between 2% and 15% by weight, relative to the total weight of the composition.

### Solid Polyester (B)

As indicated above, the composition according to the invention comprises at least one first compound that is solid at 25° C. (B), chosen from polyesters obtained at least from a monounsaturated or polyunsaturated fatty acid dimer; the fatty acid comprising from 16 to 22 carbon atoms.

More particularly, said polyesters are esters of dimer dilinoleic acid and polyol(s) or an ester thereof. The expression “or an ester thereof” is intended to denote one of the derivatives of these dimer dilinoleic acid esters of polyol(s) obtained either by reaction of alcohol function(s) of the polyol, which are not employed in bonds of ester type with acid functions of the dilinoleic acid, with one or more carboxylic functions of acid molecules other than dilinoleic acid or by reaction of acid functions of the dilinoleic dimer, which are not employed in bonds of ester type with alcohol functions of the polyol, with alcohol functions of alcohol molecules other than the polyol.

More particularly, the polyester(s) are chosen from pasty compounds. For the purposes of the present invention, the term “pasty compound” is understood to mean more particularly a lipophilic solid compound comprising, at a temperature of 25° C., a liquid fraction and a solid fraction. Thus, a pasty compound may have a starting melting point of less than 25° C.

The melting point (or melting temperature) of the pasty fatty substance can be measured using a differential scanning calorimeter (DSC), for example the calorimeter sold under the name DSC Q2000 by TA instruments. The protocol is as follows:

A sample of 5 mg of pasty fatty substance placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute.

The melting point of the pasty fatty substance is the value of the temperature corresponding to the top of the peak on the curve representing the variation in the difference in power absorbed as a function of the temperature.

It should be noted that the liquid fraction by weight of the pasty fatty substance at ambient temperature is equal to the ratio of the heat of fusion consumed at ambient temperature to the heat of fusion of the pasty fatty substance.

The heat of fusion of the pasty fatty substance is the heat consumed by said substance in order to pass from the solid state to the liquid state. The pasty fatty substance is said to be in the solid state when all of its mass is in crystalline solid form. The pasty fatty substance is said to be in the liquid state when all of its mass is in liquid form.

The heat of fusion of the pasty fatty substance is the amount of energy required to make the pasty fatty substance change from the solid state to the liquid state. It is expressed in J/g. The heat of fusion of the pasty fatty substance is equal to the area under the curve of the thermogram obtained.

### Dimer Dilinoleic Acid

The dimer dilinoleic acid that is suitable for use in the present invention may be obtained by polymerization reaction, especially by intermolecular dimerization of a linoleic acid.

The oxidation stability of the compound may be improved by hydrogenating the double bonds remaining after the dimerization reaction.

The dimer dilinoleic acid can also be obtained by dimerization of the hydrogenated form of linoleic acid.

The hydrogenated form of the acid or of the diacid may be partial or total, and may correspond, for example, to the saturated form, which is more oxidation-stable.

As indicated above, the carboxylic functions of the dimer dilinoleic acid residue not involved in the ester bond with the polyol residue(s) may be involved in other ester bonds with other alcohol functions of alcohol molecules distinct from the polyol(s).

These alcohol molecules or residues may be monoalcohols or polyols.

As examples of alcohol residues that are suitable for use in the invention, mention may be made of hydrocarbon compounds comprising a hydroxyl function and containing from 4 to 40 carbon atoms, in particular from 6 to 36 carbon atoms, in particular from 8 to 32 carbon atoms, in particular from 16 to 28 carbon atoms and more particularly from 18 to 24 carbon atoms.

As examples of monoalcohols that are suitable for the invention, mention may be made, in a non-limiting manner, of butanol, pentanol, propanol, hexanol, heptanol, octanol, decanol, dodecanol, hexadecanol, octadecanol, eicosadecanol, phytosterol, isostearol, stearol, cetol, behenol, etc.

### Polyols

The term “polyol” is intended to denote any hydrocarbon compound comprising at least two hydroxyl functions and containing from 3 to 40 carbon atoms, in particular from 6 to 36 carbon atoms, in particular from 8 to 32 carbon atoms, in particular from 16 to 28 carbon atoms and more particularly from 18 to 24 carbon atoms.

The hydrocarbon chains may be interrupted, where appropriate, by the presence of at least one heteroatom, and especially an oxygen atom.

A polyol or a polyol ester that is suitable for use in the present invention may comprise, for example, from 2 to 12 hydroxyl functions, in particular from 2 to 8 hydroxyl functions, and more particularly from 4 to 6 hydroxyl functions.

Where appropriate, the hydroxyl functions, other than those already employed in an ester bond with the dimer dilinoleic acid, may also be employed, wholly or partly with other ester bonds via reactivity with acid molecules other than the dimer dilinoleic acid.

The polyol or an ester thereof that is suitable for use in the present invention may be chosen especially from linear, branched, cyclic or polycyclic, saturated or unsaturated alcohols.

Thus, the polyol may be chosen, for example, from a diol, a triol, a tetraol, or a pentaol, or an ester thereof.

The polyol may be a diol, or an ester thereof, chosen in particular from a fatty alcohol dimer, a C₂-C₄ monoalkylene or polyalkylene glycol, 1,4-butanediol and pentaerythritol. It can also be a monoglycerol or polyglycerol or an ester thereof, or hydrogenated or non-hydrogenated castor oil (triglyceride of a hydroxylated acid).

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, 1-decanediol, tridecanediol, dodecanediol, tetradecanediol, pentadecanediol, hexadecanediol, nonadecanediol, octadecanediol, cyclohexanediol, diglycerol, erythritol, pentaerythritol, xylitol, sorbitol, ethylene glycol and xylene glycol, and isomers thereof.

A fatty alcohol dimer may also be the product of hydrogenation, for example catalytic hydrogenation, of a fatty acid dimer, which is itself obtained by dimerization of an unsaturated fatty acid, especially of C₈ to C₃₄, especially of C₁₂ to C₂₂, in particular of C₁₆ to C₂₀ and more particularly of C₁₈.

According to a particular embodiment, the fatty alcohol dimer can be a diol dimer derived from the hydrogenation of dilinoleic diacid. It is generally in a saturated form.

Preferably, the polyol is a fatty alcohol dimer such as, for example, dilinoleol dimer (dimer dilinoleyl alcohol: INCI name).

As an example of a diol that may be suitable for use in the invention, mention may be made especially of diglycerol. This compound is a glycerol dimer resulting from the condensation of two molecules of glycerol, with the loss of a water molecule. The term “diglycerol” denotes any isomer combination that can result from such a condensation, for instance linear isomers, branched isomers and, where appropriate, cyclic isomers resulting from an intramolecular dehydration of a diglycerol molecule. The diglycerol may be obtained via any process known to those skilled in the art and especially those described in patent EP 0 750 848.

As examples of acid molecules that can interact with one or more hydroxyl functions of the polyol, not employed in the ester bond with the dimer dilinoleic acid, mention may be made, in a non-limiting manner, of molecules derived from isostearic acid, behenic acid, phytosteric acid, stearic acid, hydroxystearic acid or cetylic acid.

An ester that is suitable for use in the present invention may be obtained by reacting a polyol or an ester thereof with a dimer dilinoleic acid, in a molar ratio of about 1.0:0.2-1.0.

An ester that may be suitable for use in the present invention may especially be obtained by reacting a dimer dilinoleic acid with a dilinoleol and, where appropriate, at least one additional monoalcohol chosen in particular from behenol, isostearol, sterol, in particular phytosterol, stearol, cetol, and mixtures thereof.

Thus, an ester used in the context of the present invention may be used in the form of a mixture of various esters, for example.

An ester that is suitable for the invention may be obtained, for example, by reacting a glycerol, an isostearic acid and a dimer dilinoleic acid, especially, in a molar ratio of 1.0:0.2-1.0:0.5-0.9.

As examples of esters of dimer dilinoleic acid and of polyol(s) or an ester thereof suitable for the invention, mention may be made of the esters described in applications JP2003-226609, JP2004-256515, JP2005-179377 and JP2011-020933.

The first solid or pasty compound (B) is more particularly chosen from the following compounds having the INCI name: Bis-Behenyl/Isostearyl/Phytosteryl Dimer Dilinoleyl Dimer Dilinoleate, Phytosteryl/Isosteryl/Cetyl/Stearyl/Behenyl dimer dilinoleate, Hydrogenated Castor Oil Dimer Dilinoleate, Diglycerin/Dilinoleic Acid/Hydroxystearic Acid Copolymer, and also mixtures thereof.

The bis-behenyl/isostearyl/phytosteryl dimer dilinoleyl dimer dilinoleate, and phytosteryl/isosteryl/cetyl/stearyl/behenyl dimer dilinoleate compounds are sold in particular by Nippon Fine Chemicals under the names Plandool G, Plandool H and Plandool S.

The hydrogenated castor dilinoleate, and diglycerin/dilinoleic acid/hydroxystearic acid copolymer compounds are sold in particular under the names Risocast DA-H or DA-L, or Risocast HSDA by Kokyu Alcohol Kogyo Co.

More particularly, the content of first solid or pasty compound(s) ranges from 10% to 35% by weight relative to the weight of the composition, in particular from 10% to 30% by weight, or even more particularly from 15% to 30% by weight, relative to the weight of the composition.

### Volatile or Non-Volatile First Oil (C)

The composition according to the invention also comprises at least one volatile or non-volatile first oil, chosen from apolar hydrocarbon oils; silicone oils; liquid polyesters obtained from a monounsaturated or polyunsaturated fatty acid dimer, the fatty acid comprising from 16 to 22 carbon atoms; ester, ether or carbonate hydrocarbon oils, these oils being other than the abovementioned liquid polyesters and being free of free hydroxyl groups; plant oils with the exception of castor oil; and mixtures thereof.

The term “oil” denotes lipophilic compounds that are liquid and water-immiscible at 25° C. and atmospheric pressure (1.013×10⁵ Pa).

The term “non-volatile oil” is understood to mean an oil the vapour pressure of which at 25° C. and atmospheric pressure is non-zero and is less than 2.66 Pa and more particularly less than 0.13 Pa. By way of example, the vapour pressure may be measured via the static method or via the effusion method by isothermal thermogravimetry, depending on the vapour pressure (OECD standard 104).

The term “volatile oil” is understood to mean an oil having a non-zero vapour pressure at ambient temperature and atmospheric pressure, ranging in particular from 2.66 Pa to 40 000 Pa, in particular ranging to 13 000 Pa, and more particularly ranging to 1300 Pa.

### Non-Volatile Oils

### Apolar Non-Volatile Hydrocarbon Oils

The first oil may thus be chosen from linear or branched, saturated or unsaturated, preferably saturated, non-volatile apolar hydrocarbon oils.

The linear or branched, non-volatile apolar hydrocarbon oil(s) are more particularly compounds comprising only carbon and hydrogen atoms (in other words non-volatile oils of hydrocarbon type).

Said linear or branched apolar oils may be of mineral, plant or synthetic origin, for instance:
liquid paraffin,
squalane, more particularly of plant origin,
isoeicosane,
mixtures of saturated linear hydrocarbons, more particularly of C₁₅-C₂₈, such as the mixtures the INCI names of which are, for example, the following: C15-19 Alkane (INCI name), C18-C21 Alkane (INCI name), C21-C28 Alkane (INCI name), for instance the products Gemseal 40, Gemseal 60 and Gemseal 120 sold by Total, and Emogreen L19 sold by SEPPIC,
hydrogenated or non-hydrogenated polybutenes, for instance products of the Indopol range sold by Ineos Oligomers,
hydrogenated or non-hydrogenated polyisobutenes, for instance non-volatile compounds of the Parleam® range sold by Nippon Oil Fats,
hydrogenated or non-hydrogenated polydecenes, for instance non-volatile compounds of the Puresyn® range sold by ExxonMobil,
and mixtures thereof.

### Non-Volatile Silicone Oils

The non-volatile first oil may also be chosen from phenylated or non-phenylated non-volatile silicone oils.

For the purposes of the invention, the term “silicone oil” means an oil comprising at least one silicon atom, and notably at least one Si—O group.

More particularly, said silicone oils are free of (poly)alkoxylated groups, notably such as (poly) ethoxylated or (poly) propoxylated groups, or (poly)glycerolated groups.

More particularly, the phenylated or non-phenylated non-volatile silicone oil is chosen from dimethicones, trimethylpentaphenyltrisiloxanes, tetramethyltetraphenyltrisiloxanes, diphenyl dimethicones, trimethylsiloxyphenyl dimethicones, phenyl trimethicones and diphenylsiloxyphenyl trimethicones, and also mixtures thereof.

These phenyl silicones are notably sold under the names PH-1555 HRI Cosmetic Fluid (trimethyl pentaphenyl trisiloxane) and Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu; the products Belsil PDM 1000 and Belsil PDM 20 sold by Wacker Chemie (trimethylsiloxy phenyl dimethicone), alone or as mixtures; The values in parentheses represent the viscosities at 25° C. (ASTM D-445 standard).

### Liquid Polyesters

As indicated above, the composition may comprise, as first oil (C), at least one liquid polyester obtained from an unsaturated fatty acid dimer; the fatty acid comprising from 16 to 22 carbon atoms. It should be noted that the liquid polyester is a hydrocarbon compound.

The term “liquid polyester” means a polyester which begins to flow under its own weight in less than one minute at 25° C.

In the context of the present invention, the term “unsaturated fatty acid” denotes carboxylic fatty acids comprising from 1 to 6 unsaturations (carbon-carbon double bonds).

As representatives of these unsaturated fatty acids, mention may notably be made of palmitoleic acid, oleic acid, linoleic acid, elaidic acid, gadoleic acid, eicosapentaenoic acid, docosahexaenoic acid, erucic acid, brassidic acid, arachidonic acid and mixtures thereof.

The unsaturated fatty acid dimer is more particularly obtained by an intermolecular polymerization reaction, notably a dimerization reaction, of at least one unsaturated monocarboxylic acid. It is derived in particular from the dimerization of an unsaturated fatty acid, notably a C₁₆-C₂₂ fatty acid and more particularly a C₁₈ fatty acid.

Advantageously, the liquid polyester used in the present invention is obtained from a diacid dimer obtained by dimerization of linoleic acid, optionally followed by total or partial, advantageously total, hydrogenation of the carbon-carbon bonds.

It may also be obtained from a mixture containing same. In such a case, it is preferred to use a mixture in which the content of this diacid dimer is greater than 70%, more particularly greater than 90% by weight, or even more preferentially greater than 95% by weight; the remainder of the mixture possibly being trimers and/or monomers of unsaturated fatty acids.

Furthermore, according to a first embodiment of the invention, the liquid polyester of the composition may be obtained by reacting the abovementioned acid dimer with at least one C₃-C₄ diol, which is preferably linear and preferably saturated.

Preferably, the diol is 1,2-propanediol, 1,3-propanediol, 1,2-butanediol, 1,3-butanediol or 1,4-butanediol, and preferably 1,4-butanediol.

Advantageously, the polyester used in the composition according to the invention has an average molecular weight of between 500 and 2000 g/mol, preferably between 1000 and 2000 g/mol and preferentially between 1200 and 1800 g/mol.

More particularly, the polyesters have a viscosity of between 3000 and 400 000 cSt at 40° C.

In a particularly preferred embodiment, the polyester is obtained by condensation of dilinoleic acid and 1,4-butanediol or of dilinoleic acid and 1,3-propanediol.

Mention may most particularly be made of Dilinoleic Acid/Butanediol Copolymer (INCI name), sold by Biosynthis under the name Viscoplast 14436H, or Dilinoleic Acid/Propanediol Copolymer (INCI name), sold by Biosynthis under the name Viscoplast Green 3000.

According to a second embodiment of the invention, the liquid polyester of the composition according to the invention may be obtained by reacting the abovementioned acid dimer with at least one alcohol dimer (diol dimer), which is preferably saturated, the alcohol of which is a C₁₆-C₃₂ and preferably a C₁₆-C₂₂ alcohol.

For the purposes of the present invention, the term “diol dimer” more particularly denotes saturated diols produced by hydrogenation of the corresponding diacid dimers, a diacid dimer being as defined previously.

It should be noted that the diol dimer may also be other components, for example a triol trimer, a monoalcohol and compounds of ether type, depending on the degree of purification of the dimer acid and/or of the lower alcohol ester thereof, used as starting material. It is possible to use products the diol dimer content of which is greater than 70% by weight, but it is preferred to use a diol dimer of high purity, such as a compound in which the diol dimer content is greater than 90% by weight.

Thus, a diol dimer may be produced by catalytic hydrogenation of a diacid dimer, which is itself obtained by dimerization of at least one unsaturated fatty acid, notably a C₁₆ to C₃₂ fatty acid, such as those mentioned previously, notably a C₁₆ to C₂₂ fatty acid and more particularly a C₁₈ fatty acid.

According to a particular embodiment, the diol dimer is derived from the hydrogenation of the acid functions of dilinoleic diacid.

More particularly, it is the diol dimer obtained by dimerization of linoleic acid, followed by hydrogenation of the acid functions. The diol dimer may be in the saturated form, i.e. not including any carbon-carbon double bonds. According to another embodiment, the possible carbon-carbon double bonds of the diol dimer are totally or partially hydrogenated, after esterification reaction of the diacid dimer with the diol dimer. The hydrogenated forms, notably the totally hydrogenated forms, are preferred.

As illustrations of esters that are suitable for use in the invention, mention may notably be made of the following products (INCI name): Dimer Dilinoleyl Dimer Dilinoleate, notably sold by Nippon Fine Chemical under the trade names Lusplan DD-DA5® and DD-DA7®.

Preferably, the composition according to the invention comprises, as liquid polyester (C), at least one compound (INCI names), alone or as mixtures, chosen from Dimer Dilinoleyl Dimer Dilinoleate, Dilinoleic Acid/Butanediol Copolymer and Dilinoleic Acid/Propanediol Copolymer.

### Ester, Ether or Carbonate Hydrocarbon Oils

The first oil may also be chosen from ester, ether or carbonate hydrocarbon oils, which do not have a free hydroxyl group, and which are other than the liquid polyesters obtained from an unsaturated fatty acid dimer (or liquid polyester(s)) as described above.

The term “hydrocarbon oil” or “hydrocarbon compound” conventionally denotes an oil (or a compound) formed essentially or even constituted of carbon and hydrogen atoms, and optionally oxygen or nitrogen atoms, and not containing a silicon or fluorine atom. The hydrocarbon oil is thus different from a silicone oil and a fluoro oil. Advantageously, the hydrocarbon oils according to the invention contain only carbon, hydrogen, oxygen and possibly nitrogen atoms.

According to a first embodiment, the first oil is chosen from ester oils, comprising 1 to 4 ester functions, comprising in total between 17 and 80 carbon atoms, more particularly between 17 and 70 carbon atoms, which are linear or branched, and saturated, unsaturated or aromatic.

More particularly, said esters are chosen from monoesters and polyesters, other than the abovementioned liquid polyesters, for instance:
saturated or unsaturated monoesters, of a monocarboxylic acid and of a monoalcohol, for instance purcellin oil (cetostearyl octanoate), isononyl isononanoate, C₁₂ to C₁₅ alkyl benzoate, octyldodecyl neopentanoate, 2-octyldodecyl stearate, 2-octyldodecyl erucate, oleyl erucate, isostearyl isostearate, 2-octyldodecyl benzoate, isopropyl myristate, isopropyl palmitate, butyl stearate, hexyl laurate, 2-ethylhexyl palmitate, 2-hexyldecyl laurate, 2-octyldecyl palmitate and 2-octyldodecyl myristate;
diesters, for instance diesters of dicarboxylic acids and of monoalcohols, or diesters of glycol and of monocarboxylic acids, such as neopentyl glycol diheptanoate, propylene glycol dioctanoate or diethylene glycol diisononanoate;
triesters of a tricarboxylic acid and of a monoalcohol, such as triisostearyl citrate or tridecyl trimellitate;
total esters of glycerol, of diglycerol, of pentaerythritol and of monocarboxylic acids, for instance heptanoic or octanoic acid triglycerides, capric acid triglycerides alone or as a mixture, for instance capric/caprylic acid triglycerides, C18-36 acid triglycerides; 2-decyl triglyceride tetradecanoate, pentaerythrityl tetrapelargonate, pentaerythrityl tetraisostearate, pentaerythrityl tetraisononanoate, polyglyceryl-2 tetraisostearate; Pentaerythrityl Isostearate/Caprate/Caprylate/Adipate (INCI name, for example the Supermol L products from Croda);
and also mixtures thereof.

According to a second embodiment of the invention, the non-volatile first oil may be chosen from the ethers of formula ROR′, the carbonates of formula RO(CO)OR′, in which formulae the groups R and R′, which may be identical or different, represent a saturated or unsaturated, branched or unbranched hydrocarbon group comprising less than 16 carbon atoms, preferably a C₃-C₁₆ group. For example, mention may be made of dicaprylyl ether, dipropyl carbonate, diethylhexyl carbonate, dicaprylyl carbonate, C14-15 dialkyl carbonate, and also mixtures thereof.

### Plant Oils

The first oil may likewise be chosen from plant oils with the exception of castor oil and of lesquerella oil, preferably free of free hydroxyl group(s), and mixtures thereof.

Examples of plant oils that may be mentioned include those chosen from jojoba oil, olive oil, coconut oil, ximenia oil, pracaxi oil, coriander seed oil, macadamia oil, passionflower oil, argan oil, sesame oil, sunflower oil, grape seed oil, avocado oil, dog rose oil, apricot kernel oil, linseed oil, sweet almond oil, cottonseed oil, soybean oil, rapeseed oil, canola oil, groundnut oil, kaya oil, wheat germ oil, corn oil, alfalfa oil, poppy oil, pumpkin oil, marrow oil, hazelnut oil, blackcurrant oil, evening primrose oil, millet oil, barley oil, quinoa oil, rye oil, safflower oil, candlenut oil, passionflower oil, the liquid fraction of shea butter, and the liquid fraction of cocoa butter, and also mixtures thereof.

Preferably, the plant oil is chosen from sunflower oil, olive oil, apricot oil, sweet almond oil, argan oil, rapeseed oil, canola oil, jojoba oil and sesame seed oil, alone or as mixtures.

According to a preferred variant of the invention, the non-volatile first oil (C) is chosen from non-volatile apolar hydrocarbon oils, from liquid polyesters, from ester hydrocarbon oils other than the abovementioned liquid polyesters, comprising 1 to 4 ester functions, comprising in total between 17 and 70 carbon atoms, which are linear or branched, and saturated, unsaturated or aromatic, free of free hydroxyl groups, from plant oils with the exception of castor oil and lesquerella oil; dicaprylyl ether and dicaprylyl carbonate, alone or as mixtures.

Preferably, the non-volatile first oil (C) is chosen from squalane, more particularly of plant origin, dimer dilinoleyl dimer dilinoleate, dilinoleic acid/butanediol copolymer, dilinoleic acid/propanediol copolymer, caprylic/capric triglyceride, oleyl erucate, pentaerythrityl tetraisostearate, olive oil, sunflower oil, apricot oil, sweet almond oil, argan oil, rapeseed oil, canola oil, jojoba oil, sesame oil, alone or as mixtures.

### Volatile Oils

The composition may also comprise, as first oil(s), at least one volatile hydrocarbon or silicone oil, and also mixtures thereof.

The hydrocarbon volatile oils are preferably chosen from hydrocarbon oils of hydrocarbon type (thus apolar hydrocarbon oils, constituted solely of carbon and hydrogen). In particular, they may be chosen from volatile hydrocarbon oils containing from 8 to 16 carbon atoms, and mixtures thereof, and notably:
branched C₈-C₁₆ alkanes, such as isoalkanes (also known as isoparaffins), isododecane, isodecane or isohexadecane, and mixtures thereof, and for example the oils sold under the Isopar or Permethyl trade names,
linear alkanes, for example C₁₁-C₁₅ alkanes, alone or as mixtures, and
mixtures thereof.

The volatile silicone oils can be chosen from linear, branched or cyclic silicone oils such as polydimethylsiloxanes (PDMS) containing from 3 to 7 silicon atoms, preferably linear or branched polydimethylsiloxanes containing from 3 to 7 silicon atoms, and mixtures thereof.

Examples of such oils that may be mentioned include octyl trimethicone, hexyl trimethicone, methyl trimethicone, decamethylcyclopentasiloxane, octamethylcyclotetrasiloxane, dodecamethylcyclohexasiloxane, decamethyltetrasiloxane, polydimethylsiloxanes such as those sold under the reference DC 200 (1.5 cSt), or DC 200 (3 cSt) by Dow Corning or KF 96 A from Shin-Etsu; alone or as mixtures.

If the composition comprises at least one volatile oil, said oil is more particularly chosen from volatile hydrocarbon oils, preferably hydrocarbon oils.

According to a preferred embodiment, the first oil (C) comprises at least one non-volatile oil, in particular chosen from the following non-volatile oils:
apolar hydrocarbon oils chosen from liquid paraffin, squalane, more particularly of plant origin, isoeicosane, C₁₅₋₁₉ alkane, C₁₈₋₂₁ alkane, C₂₁₋₂₈ alkane, hydrogenated or non-hydrogenated polybutenes; hydrogenated or non-hydrogenated polyisobutenes, hydrogenated or non-hydrogenated polydecenes, and also mixtures thereof;
non-phenylated silicone oils chosen from dimethicones, from phenylated non-volatile silicones chosen from trimethyl pentaphenyl trisiloxane, tetramethyl tetraphenyl trisiloxane, diphenyl dimethicones, trimethylsiloxyphenyl dimethicones, phenyltrimethicone and diphenylsiloxyphenyl trimethicone, and mixtures thereof;
liquid polyesters obtained from dilinoleic dimer acid, and from at least one C₃-C₄ diol; from an alcohol dimer, which is preferably saturated, the alcohol of which is C₁₆-C₃₂, preferably C₁₆-C₂₂; and more particularly, from at least propanediol, butanediol, C₁₈-C₃₀ alcohols, dilinoleyl alcohol,
hydrocarbon ester oils, other than the abovementioned liquid polyesters, comprising 1 to 4 ester functions, comprising in total between 17 and 70 carbon atoms, which are linear or branched, and saturated, unsaturated or aromatic, free of free hydroxyl groups,
plant oils with the exception of castor oil and of lesquerella oil,
dicaprylyl ether,
dicaprylyl carbonate,
and also mixtures thereof.

According to an even more particular embodiment, the first oil (C) comprises at least one non-volatile oil chosen from the following non-volatile oils:
apolar hydrocarbon oils chosen from liquid paraffin, squalane, more particularly of plant origin, isoeicosane, C₁₅₋₁₉ alkane, C₁₈₋₂₁ alkane, C₂₁₋₂₈ alkane, hydrogenated or non-hydrogenated polybutenes; hydrogenated or non-hydrogenated polyisobutenes, hydrogenated or non-hydrogenated polydecenes, and also mixtures thereof;
liquid polyesters obtained from dilinoleic dimer acid, and from at least one C₃-C₄ diol; from an alcohol dimer, which is preferably saturated, the alcohol of which is C₁₆-C₃₂, preferably C₁₆-C₂₂; and more particularly, from at least propanediol, butanediol, C₁₈-C₃₀ alcohols, dilinoleyl alcohol,
hydrocarbon ester oils, other than the abovementioned liquid polyesters, comprising 1 to 4 ester functions, comprising in total between 17 and 70 carbon atoms, which are linear or branched, and saturated, unsaturated or aromatic, free of free hydroxyl groups,
plant oils with the exception of castor oil and of lesquerella oil,
and also mixtures thereof.

Preferably, the first oil (C) comprises at least one non-volatile oil chosen from squalane, more particularly of plant origin, Dimer Dilinoleyl Dimer Dilinoleate (INCI name), dilinoleic acid/butanediol copolymer (INCI name), dilinoleic acid/propanediol copolymer (INCI name), capric/caprylic acid triglycerides, oleyl erucate, pentaerythrityl tetraisostearate, olive oil, sunflower oil, apricot oil, sweet almond oil, argan oil, rapeseed oil, canola oil, jojoba oil, sesame oil, and also mixtures thereof.

More particularly, the content of first oil(s) is between 40 and 60% by weight, more particularly between 45 and 55% by weight, relative to the weight of the composition.

In accordance with an advantageous embodiment of the invention, the content of non-volatile first oil(s) is between 40% and 60% by weight, more particularly between 45% and 55% by weight, relative to the total weight of the composition.

Moreover, if the composition comprises same, the content of volatile first oil(s) is less than or equal to 15% by weight and more particularly less than or equal to 10% by weight, relative to the total weight of the composition. Preferably, the composition does not comprise any volatile oil.

### Lipophilic Thickener

The composition according to the invention may also comprise at least one lipophilic thickener, chosen from silicas, which may or may not have undergone a hydrophobic treatment; dextrin esters, alone or as a mixture.

### Silicas

The composition according to the invention may also comprise, as lipophilic thickener, a fumed silica, preferably a hydrophobic fumed silica, or silica aerogel particles, preferably hydrophobic silica aerogel particles.

The term “hydrophobic silica” means any silica of which the surface is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

### a) Fumed Silica

Fumed silica which has undergone a hydrophobic surface treatment is particularly suitable for use in the invention. It is in fact possible to chemically modify the surface of the silica, by chemical reaction generating a reduction in the number of silanol groups present at the surface of the silica. It is notably possible to substitute silanol groups with hydrophobic groups: a hydrophobic silica is then obtained.

The hydrophobic groups may be:
trimethylsiloxyl groups, which are notably obtained by treating fumed silica in the presence of hexamethyldisilazane. Silicas thus treated are known as “Silica silylate” according to the CTFA (8th edition, 2000). They are sold, for example, under the references Aerosil R812® by Degussa, and Cab-O-Sil TS-530® by Cabot.
dimethylsilyloxyl or polydimethylsiloxane groups, which are notably obtained by treating fumed silica in the presence of polydimethylsiloxane or dimethyldichlorosilane. Silicas thus treated are known as “Silica dimethyl silylate” according to the CTFA (8th Edition, 2000). They are sold, for example, under the references Aerosil R972® and Aerosil R974® by Degussa and Cab-O-Sil TS-610® and Cab-O-Sil TS-720® by Cabot.

### b) Silica Aerogels

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogel particles usually have a specific surface area per unit mass (S_{M}) ranging from 500 to 1500 m²/g, preferably from 600 to 1200 m²/g and better still from 600 to 800 m²/g, and a size expressed as the volume mean diameter (D [0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles used in the present invention have a size expressed as volume mean diameter (D [0.5]) ranging from 1 to 30 μm, preferably from 5 to 25 μm, better still from 5 to 20 μm and even better still from 5 to 15 μm.

The specific surface per unit of weight can be determined by the nitrogen absorption method, known as the BET (Brunauer-Emmett-Teller) method, described in The Journal of the American Chemical Society, Vol. 60, page 309, February 1938, and corresponding to the international standard ISO 5794/1 (Annex D). The BET specific surface area corresponds to the total specific surface area of the particles under consideration.

The sizes of the silica aerogel particles may be measured by static light scattering using a commercial particle size analyzer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H. C., Light Scattering by Small Particles, Chapters 9 and 10, Wiley, New York, 1957.

According to one advantageous embodiment, the hydrophobic silica aerogel particles used in the present invention have a specific surface area per unit mass (S_{M}) ranging from 600 to 800 m²/g and a size expressed as the volume mean diameter (D [0.5]) ranging from 5 to 20 μm and even better still from 5 to 15 μm.

The aerogels are aerogels of hydrophobic silica, preferably of silylated silica (INCI name: silica silylate).

As regards the preparation of hydrophobic silica aerogel particles that have been surface-modified by silylation, reference may be made to U.S. Pat. No. 7,470,725.

Use will preferably be made of hydrophobic silica aerogel particles surface-modified with trimethylsilyl groups.

As hydrophobic silica aerogels that may be used in the invention, examples that may be mentioned include the aerogel sold under the name VM-2260 (INCI name: Silica silylate), by Dow Corning, the particles of which have an average size of about 1000 microns and a specific surface area per unit mass ranging from 600 to 800 m²/g.

Mention may also be made of the aerogels sold by Cabot under the references Aerogel TLD 201, Aerogel OGD 201, Aerogel TLD 203, Enova® Aerogel MT 1100 and Enova Aerogel MT 1200.

Use will preferably be made of the aerogel sold under the name VM-2270 (INCI name: Silica silylate) by Dow Corning, the particles of which have an average size ranging from 5-15 μm and a specific surface area per unit of mass ranging from 600 to 800 m²/g.

### Dextrin Esters

According to another embodiment, the lipophilic thickener is chosen from dextrin esters, more particularly a fatty acid ester of dextrin, which is preferably a C₈-C₃₀ ester.

More particularly, the fatty acid ester of dextrin corresponds to formula (I):

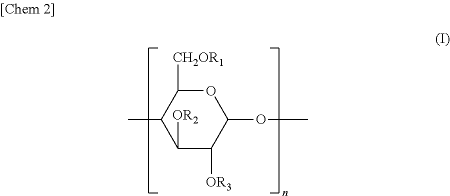

in which:
the radicals R₁, R₂ and R₃, which may be identical or different, are chosen from hydrogen or an acyl group (R—CO—) in which the radical R is a linear or branched, saturated or unsaturated hydrocarbon group containing from 6 to 30, notably from 8 to 22, or even 12-18 carbon atoms, with the proviso that at least one of said radicals R₁, R₂ or R₃ is other than hydrogen,
n is an integer between 3 and 150, notably 10 and 100 and preferably 15 and 40.

Preferably, the three radicals R₁, R₂ and R₅ are other than hydrogen, thereby resulting in dextrin esters of formula (II) below:

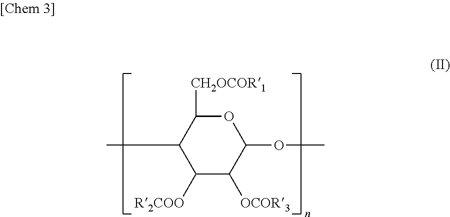

in which the radicals R′₁, R′₂ and R′₃, which may be identical or different, represent a linear or branched, saturated or unsaturated, hydrocarbon group containing 6 to 30, notably 8 to 22, more particularly 12-18 carbon atoms, —n is an integer between 3 and 150, notably 10 and 100, and preferably 15 and 40.

Notably, the radicals —OCOR′₁, —OCOR′₂ and/or —OCOR′₃ may be chosen from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoarachic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic and stearolic radicals, and mixtures thereof.

Preferably, the dextrin ester is chosen from dextrin palmitate and dextrin myristate. Some of these dextrin esters are commercially available, notably under the name Rheopearl from Chiba Flour Milling Co.

When the composition comprises any, the content of lipophilic thickener(s) more particularly represents from 0.1% to 10% by weight, preferably from 0.5% to 5% by weight, relative to the total weight of the composition.

Preferably, if the composition comprises silica, said silica is chosen from fumed silica, which is preferably hydrophobic, or silica aerogel particles, which are preferably hydrophobic.

According to another particular embodiment of the invention, the composition according to the invention may comprise at least one lipophilic thickener chosen from natural or synthetic clays, which have in particular been made lipophilic by treatment with an alkyl ammonium salt, for instance a C₁₀ to C₂₂ chloride, for example distearyldimethylammonium chloride.

They may be chosen from bentonites, in particular hectorites and montmorillonites, beidellites, saponites, nontronites, sepiolites, biotites, attapulgites, vermiculites and zeolites. They are preferably chosen from hectorites.

More particularly, the clays are lipophilic and chosen in particular from hectorites modified with a C₁₀ to C₂₂ ammonium halide, notably chloride, such as hectorite modified with distearyldimethylammonium chloride, for instance the product sold under the name Bentone 38V® by Elementis or bentone gel in isododecane sold under the name Bentone Gel ISD V® (87% isododecane/10% disteardimonium hectorite/3% propylene carbonate) by Elementis.

If the composition according to the invention were to comprise such clays, their content would preferably be not greater than 2% by weight, preferably not greater than 1% by weight, relative to the total weight of the composition.

According to a preferred embodiment of the invention, the composition according to the invention does not comprise any modified or unmodified clay.

### Water

The composition according to the invention may optionally comprise water.

If the composition comprises any, its content is less than or equal to 10% by weight, more particularly less than or equal to 8% by weight. Advantageously, the content of water, if it is present, is between 0.05% and 8% by weight and more particularly between 0.1% and 5% by weight relative to the total weight of the composition.

### Hydroxylated Solvents

The composition according to the invention may optionally comprise at least one hydroxylated solvent, more particularly chosen from monoalcohols containing from 1 to 5 carbon atoms, notably such as ethanol, isopropanol, and also mixtures thereof.

The hydroxylated solvent may also be chosen from C₂-C₈ polyols that are liquid at ambient temperature and atmospheric pressure, comprising from 2 to 4 free hydroxyl groups. Examples that may be mentioned include ethylene glycol, propylene glycol, 1,3-propanediol, butylene glycol, 1,3-butylene glycol, isoprene glycol, pentylene glycol, hexylene glycol, caprylyl glycol, pentaerythritol, trimethylolpropane, dipropylene glycol, glycerol, diglycerol, and mixtures thereof.

If the composition contains any, then the content of hydroxylated solvent(s) is less than or equal to 10% by weight, more particularly less than or equal to 8% by weight. Advantageously, if the composition contains any, the content of hydroxylated solvent(s) is between 0.05% and 8% by weight, more particularly between 0.1% and 5% by weight, relative to the total weight of the composition.

It should be noted that if the composition comprises water, then the amount of water and of hydroxylated solvent(s) is less than or equal to 10% by weight, more particularly less than or equal to 8% by weight and even more preferentially less than or equal to 5% by weight, relative to the total weight of the composition.

### Solid Fatty Compounds

The composition according to the invention may optionally comprise at least one solid fatty compound other than the compounds (B) above, and more particularly chosen from waxes, pasty fatty compounds, and mixtures thereof.

### Waxes

As indicated previously, the composition according to the invention may comprise at least one wax.

The term “waxes” means water-insoluble solid fatty compounds, in particular having a melting point of between 4° and 120° C., more advantageously between 45 and 100° C. and even more preferentially from 50 to 100° C.

For the purposes of the invention, the melting point corresponds to the temperature of the most endothermic peak observed on thermal analysis (DSC) as described in the ISO 11357-3; 1999 standard. The melting point of the wax may be measured using a differential scanning calorimeter (DSC), for example the calorimeter sold under the name DSC Q2000 by TA Instruments.

The measuring protocol is as follows:

A sample of approximately 5 mg of wax is placed in a “hermetic aluminium capsule” crucible.

The sample is subjected to a first temperature rise passing from −20° C. to 120° C., at a heating rate of 10° C./minute, it is then cooled from 120° C. to −20° C. at a cooling rate of 10° C./minute and is finally subjected to a second temperature rise passing from −20° C. to 120° C. at a heating rate of 5° C./minute. During the second temperature rise, the melting point value of the solid fatty substance is measured, which corresponds to the value of the top of the most endothermic peak observed on the melting curve, representing the variation in the difference in power absorbed as a function of the temperature.

More particularly, the waxes are chosen from apolar hydrocarbon waxes, ester hydrocarbon waxes, and also mixtures thereof.

Among the apolar hydrocarbon waxes (i.e. waxes comprising only carbon and hydrogen atoms in their structure), mention may notably be made of polyethylene waxes, microcrystalline waxes, paraffin waxes, ozokerite, polymethylene waxes, waxes obtained by Fischer-Tropsch synthesis, microwaxes, notably of polyethylene, and also mixtures thereof.

Among the ester hydrocarbon waxes, mention may be made of fatty acid monoesters, dicarboxylic acid diesters, total esters of glycerol and of a C₁₆-C₃₀ carboxylic acid, these waxes not comprising any free hydroxyl groups; waxes of animal or plant origin with the exception of carnauba wax; waxes obtained by hydrogenation of animal or plant oils, with the exception of hydrogenated castor oil and of hydrogenated lesquerella oil; and also mixtures thereof.

More particularly, mention may be made of waxes of fatty acid esters of formula R₁COOR₂ in which R₁ and R₂ represent linear, branched or cyclic aliphatic chains in which the number of atoms ranges from 10 to 50, which may contain a heteroatom, in particular oxygen. In particular, cetyl palmitate and C₂₀-C₄₀ alkyl stearates may be used as wax. Such waxes are notably sold under the names Kester Wax® K82H by Koster Keunen. Use may also be made of mixtures of esters of C₁₄-C₁₈ carboxylic acids and of alcohols, such as the products Cetyl Ester Wax 814 from Koster Keunen, SP Crodamol MS MBAL or Crodamol MS PA from Croda, or Miraceti from Laserson.

Diesters of a dicarboxylic acid of general formula R₃—(—OCO—R₄—COO—R₅), in which R₃ and R₅ are identical or different, preferably identical, and represent a C₄-C₃₀ alkyl group and R₄ represents a linear or branched C₄-C₃₀ aliphatic group which may or may not contain one or more unsaturations, are also suitable for use as ester waxes. Preferably, the C₄-C₃₀ aliphatic group is linear and unsaturated.

Use may also be made of total esters of a saturated C₁₆-C₃₀ carboxylic acid, with glycerol, for instance tristearin (or glyceryl tristearate) or tribehenin (or glyceryl tribehenate), alone or as a mixture.

The waxes of animal or plant origin, with the exception of carnauba wax, for instance beeswax, synthetic beeswax, candelilla wax, lanolin wax, rice bran wax, ouricury wax, esparto grass wax, berry wax, shellac wax, cork fibre wax, sugar cane wax, Japan wax, sumac wax, montan wax, orange and lemon waxes, laurel wax, hydrogenated jojoba wax or sunflower wax, in particular refined sunflower wax.

It would also be possible to use at least one wax chosen from those obtained by hydrogenation of animal or plant oils, with the exception of optionally esterified hydrogenated castor oil and of hydrogenated lesquerella oil.

Mention may more particularly be made of the waxes obtained by catalytic hydrogenation of plant oils having linear or branched C₈-C₃₂ fatty chains, for example such as hydrogenated sunflower oil, hydrogenated olive oil, hydrogenated coconut oil or hydrogenated jojoba oil.

As waxes resulting from the hydrogenation of esters obtained from C6-C22 fatty alcohols of plant origin and plant oil, mention may also be made of the waxes with the following INCI names: Hydrogenated Olive Oil Lauryl esters, Hydrogenated Olive Oil Myristyl Esters, Hydrogenated Olive Oil Cetyl Esters, Hydrogenated Olive Oil Stearyl Esters. Such waxes are sold in particular under the names Phytowax® Olive 12 L44, Phytowax® Olive 14 L 48, Phytowax® Olive 16 L 55, Phytowax® Olive 18 L 57, by Sophim. Such waxes are described in patent application FR 2 792 190.

Preferably, if the composition comprises at least one wax, said wax is preferably chosen from beeswax, rice bran wax, sunflower wax, fatty acid ester waxes, in particular cetyl palmitate, polyethylene wax, Fischer-Tropsch waxes, ozokerite, and also mixtures thereof.

If the composition comprises any, the content of wax(es) is between 8% and 20% by weight, more particularly between 10% and 15% by weight, relative to the total weight of the composition.

### Pasty Compounds

The composition according to the invention may also comprise at least one pasty compound, other than the first solid compound(s) (B), more particularly an apolar or polar hydrocarbon compound.

Among the pasty compounds that may be used in the composition according to the invention, mention may be made of petroleum jelly (INCI name: Petrolatum) among the apolar hydrocarbon compounds (in other words compounds comprising only carbon and hydrogen atoms in their structure).

Among the polar pasty hydrocarbon compounds, use is made more particularly of compounds of ester type.

Examples that may be mentioned include lanolins and derivatives thereof, for instance acetylated lanolins or isopropyl lanolate, and mixtures thereof.

Butters of plant origin, for instance shea butter, cocoa butter, mango butter, cupuacu butter, murumuru butter, jojoba butter, and also mixtures thereof, are also suitable for use.

Mention may similarly be made of esters derived from pentaerythritol, for instance the products of INCI name Dipentaerythrityl Tetrahydroxystearate/Tetraisostearate; triglycerides of saturated fatty acids or of hydrogenated fatty acids, for instance the products of INCI name Caprylic/Capric/Myristic/Stearic Triglyceride, Hydrogenated Cocoyl Glycerides, and mixtures thereof.

Totally or partially hydrogenated plant oils may also be used as pasty compounds, with the exception of optionally esterified hydrogenated castor oil and of hydrogenated lesquerella oil, such as for example hydrogenated soybean oil, hydrogenated coconut oil, hydrogenated rapeseed oil, partially hydrogenated olive oil, and mixtures thereof.

According to a preferred embodiment of the invention, the pasty compound(s) are chosen from plant butters; totally or partially hydrogenated, preferably partially hydrogenated, plant oils, with the exception of optionally esterified hydrogenated castor oil and of hydrogenated lesquerella oil; dipentaerythrityl tetrahydroxystearate/tetraisostearate, caprylic/capric/myristic/stearic triglyceride; and mixtures thereof.

If the composition comprises any, the content of pasty fatty compound(s) other than the first solid compound(s) (B) is between 0.5% and 15% by weight, more particularly between 1% and 10% by weight, relative to the total weight of the composition.

### Additional Compounds

The composition according to the invention may optionally comprise at least one additional compound chosen from oils, waxes, pasty compounds other than solid compounds (B), first oils (C), and also solid fatty compounds, waxes or pasty fatty compounds, detailed above.

More particularly, said additional compound(s) are chosen from C₈-C₆₀ fatty alcohols; hydroxylated esters; hydrogenated or non-hydrogenated, optionally esterified castor oil; hydrogenated or non-hydrogenated lesquerella oil; carnauba wax; glyceryl trihydroxystearate (INCI name Trihydroxystearin); mono-oxyalkylenated or poly-oxyalkylenated hydrocarbon or silicone compounds, the alkyl group(s) of which is/are of C₂-C₃, mono-glycerolated or poly-glycerolated hydrocarbon or silicone compounds, bis-diglyceryl polyacyladipate-2, and also mixtures thereof.

Among the saturated or unsaturated C₈-C₃₀ alcohols, more particularly monoalcohols, which are liquid or solid at ambient temperature, mention may be made of lauryl alcohol, myristyl alcohol, cetyl alcohol, stearyl alcohol, isostearyl alcohol, oleyl alcohol, cetearyl alcohol (mixture of cetyl and stearyl alcohols), behenyl alcohol, erucyl alcohol, arachidyl alcohol, 2-hexyldecyl alcohol, isocetyl alcohol, octyldodecanol, 1-triacontanyl alcohol, and also mixtures of long-chain fatty alcohols, for instance mixtures of C₃₀-C₅₀ alcohols, C₂₀-C₄₀ alcohols and mixtures thereof.

Among the hydroxylated esters, mention may be made of hydroxylated monoesters and diesters, preferably with a total carbon number ranging from 20 to 70, for instance isostearyl lactate, octyl hydroxystearate, octyldodecyl hydroxystearate or diisostearyl malate. Examples that may also be mentioned include partial esters of glycerol or polyglycerol comprising from 2 to 8 glycerol units, for instance glyceryl stearate, polyglyceryl-2 diisostearate, polyglyceryl-3 diisostearate, polyglyceryl-3 polyricinoleate, polyglyceryl-6 polyricinoleate, polyglyceryl-4 diisostearate/polyhydroxystearate/sebacate, and also mixtures thereof. Such esters may optionally be oxyethylenated and/or oxypropylenated.

Mention may also be made of waxes resulting from the hydrogenation of esters obtained from C₆-C₂₂ fatty alcohols of plant origin and plant oil, such as the products with the following INCI names: Hydrogenated Cetyl Castor Esters, Hydrogenated Stearyl Castor Esters, Hydrogenated Castor Esters, sold in particular under the names Phytowax® Ricin 16 L 64, Phytowax® Ricin 18 L 69 and Phytowax® Ricin 22 L 73 by Sophim. Such waxes are also described in application FR2792190.

Among the pasty compounds, mention may also be made of bis-Diglyceryl polyacyladipate-2 (INCI name), in particular sold under the trademark Softisan 649 by Sasol.

Preferably, the composition according to the invention does not comprise any additional compounds.

If, however, the composition were to comprise any, their content would preferably be less than 7% by weight, more particularly less than 5% by weight, relative to the total weight of the composition. More precisely, this content would be less than 3% by weight, relative to the total weight of the composition.

Advantageously, the content of liquid additional compound(s) is less than that of first oil(s) (C) described above.

Advantageously, the content of additional solid compound(s) remains less than that of the solid compounds (B) described above, if the composition comprises these two types of compounds.

In accordance with an advantageous embodiment of the invention, the overall content of water and/or of hydroxylated solvent(s) and/or of additional compound(s), if one or more are present, is less than or equal to 10% by weight, more particularly less than or equal to 8% by weight, relative to the total weight of the composition.

### Additional Dyestuffs

The composition according to the invention may optionally comprise, in addition to the natural dyestuffs or dyestuffs of natural origin (A) described previously, at least one additional dyestuff, which is preferably solid.

More particularly, the optional dyestuff may be chosen from pearlescent agents or pigments.

### Pearlescent Agents

The term “pearlescent agents” should be understood as meaning coloured particles of any form, which may or may not be iridescent, notably produced by certain molluscs in their shell, or alternatively synthesized, and which have a colour effect via optical interference.

The pearlescent agents may be chosen from pearlescent pigments such as titanium mica coated with an iron oxide, titanium mica coated with bismuth oxychloride, titanium mica coated with chromium oxide, titanium mica coated with an organic dye and also pearlescent pigments based on bismuth oxychloride. They may also be mica particles, at the surface of which are superposed at least two successive layers of metal oxides and/or of organic dyes.

Examples of pearlescent agents that may also be mentioned include natural mica covered with titanium oxide, with iron oxide, with natural pigment or with bismuth oxychloride.

The pearlescent agents can more particularly have a yellow, pink, red, bronze, orangey, brown, gold and/or coppery colour or tint.

Among the pearlescent agents available on the market, mention may be made of the Timica, Flamenco, Cloisonne, Chromalite and Duochrome pearlescent agents (based on mica) sold by Engelhard, the Timiron, Colorona and Microna pearlescent agents sold by Merck, the Prestige mica-based pearlescent agents, sold by Eckart, and the Sunshine synthetic mica-based pearlescent agents, sold by Sun Chemical.

According to an advantageous embodiment of the invention, the pearlescent agents are chosen from those with a mean particle size (corresponding to the D50 (mean diameter, measured by laser particle size analysis or other equivalent method known to those skilled in the art)) of at least 70 μm, preferably of at least 100 μm.

As a guide, if the composition contains any, such pearlescent agents may be present in a content ranging from 0.001% to 20% by weight, in particular between 0.01% and 15% by weight, relative to the total weight of the composition.

### Pigments

The composition according to the invention may optionally comprise at least one pigment.

The term “pigments” means white or coloured, mineral and/or organic particles, which are insoluble in an aqueous or oily medium, and which are intended to colour and/or opacify the resulting composition and/or deposit.

These pigments may be coated or uncoated mineral pigments.

Among the mineral pigments that are useful in the present invention, mention may be made of zirconium oxide or cerium oxide, titanium oxides and also zinc oxide, iron oxide (black, yellow or red) or chromium oxide, manganese violet, ultramarine blue, chromium hydrate and ferric blue, titanium dioxide, and metal powders, for instance aluminium powder and copper powder.

These pigments can also be organic pigments.

The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

These pigments may also be in the form of composite pigments as described in patent EP 1 184 426. These composite pigments may be composed in particular of particles comprising an inorganic core at least partially covered with an organic pigment and at least one binder providing the fixing of the organic pigments to the core.

The pigment can also be a lake. The term “lake” means insolubilized dyes adsorbed or precipitated onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Advantageously, the pigments may have been subjected to a hydrophobic surface treatment.

The hydrophobic treatment agent may be chosen, for example, from silicones such as methicones, dimethicones and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, amino acids; N-acylamino acids or salts thereof; lecithin, isopropyl triisostearyl titanate, and mixtures thereof.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be the aluminium, magnesium, calcium, zirconium, zinc, sodium or potassium salts. The amino acid may be, for example, lysine, glutamic acid or alanine.

The term “alkyl” mentioned in the compounds cited previously notably denotes an alkyl group containing from 1 to 30 carbon atoms and preferably containing from 5 to 16 carbon atoms.

Hydrophobic treated pigments are notably described in patent application EP-A-1 086 683.

Mention may be made, among the organic dyes, of cochineal carmine.

Mention may also be made of the products, and also the lakes thereof, known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

According to a preferred embodiment of the invention, the pigment(s) have been subjected to a hydrophobic treatment.

As a guide, if the composition comprises any, the content of pigments may range from 0.001% to 10% by weight, notably from 0.01% to 5% by weight, relative to the total weight of said composition.

Advantageously, the content of additional dyestuffs is such that the composition according to the invention shows a variation of colour under the extreme conditions detailed below:

### Protocol:

The two samples below are prepared:

### Sample 1

2.5 g of composition according to the invention
0.5 g octyldodecanol
1 g of pH 10 buffer solution

The composition is crushed with a spatula and mixed with the rest of the abovementioned ingredients for one minute at 3000 rpm (for example by means of a speed mixer).

### Sample 2

2.5 g of composition according to the invention
0.5 g octyldodecanol
1 g of solution of water+citric acid at pH 3

The composition is crushed with a spatula and mixed with the rest of the abovementioned ingredients for one minute at 3000 rpm (for example by means of a speed mixer).

### Measurement

One hour after the preparation of each sample, an appropriate amount of mixture is spread as a deposit of uniform thickness using a Multiple Clearance Square Applicator 5363, sold by Byk-Gardner (thickness 2 mils, i.e. 51 μm) onto the white side of a contrast card (unvarnished Byk card, ref. 2831).
The colour of the deposit of Sample 1 on the one hand, and of Sample 2 on the other hand, is measured after 24 hours by means of a CM600d colorimeter from Konica Minolta in the CIELab 76 system (illuminant D65, SCI, 10° Observer) at three different locations away from the edges of the deposit, and the L*, a* and b* coordinates of the colour are obtained in the CIELab 76 space.
The mean of each L*, a*, b* coordinate for each sample is then calculated.
The variation in colouration is evaluated from the calculation of the mean ΔE₂₀₀₀ according to the ISO 11664-6:2014 standard (specification of the method of calculation of the colour differences according to the formula CIEDE2000).

Thus, the contents of additional dyestuffs are more particularly such that, under these extreme test conditions (not representative of the usual use of a composition for making up the skin or the lips), the variation of mean ΔE₂₀₀₀ is greater than 2 in absolute value.

### Fillers

The composition according to the invention may optionally comprise one or more filler(s) conventionally used in makeup and/or care compositions. It should be noted that the fillers are neither pigments nor pearlescent agents. More particularly, the fillers are not capable of colouring the composition and the resulting deposit.

These fillers are colourless or white solid particles of any form, which are in a form that is insoluble and dispersed in the medium of the composition.

These fillers, of mineral or organic, natural or synthetic nature, give the composition containing them softness and give the makeup result a matt effect and uniformity. In addition, these fillers advantageously make it possible to combat various attacking factors such as sebum or sweat.

By way of illustration of these fillers, mention may be made of mica, silica, kaolin, poly-β-alanine and polyethylene powders, tetrafluoroethylene polymer (Teflon®) powders, lauroyl-lysine, starch, celluloses such as the products of the Cellulobeads range sold by Daito Kasei Kogyo, the products of the Sensocel range, in particular Sensocel 8, sold by Rossow, rice powders (in particular sold by Daito Kasei Kogyo), boron nitride, polymeric hollow microspheres such as those of polyvinylidene chloride/acrylonitrile such as Expancel® (Nobel Industrie), or of acrylic acid copolymers, silicone resin microbeads (Tospearls® from Toshiba, for example), elastomeric polyorganosiloxane particles, precipitated calcium carbonate, magnesium carbonate and magnesium hydrogen carbonate, hydroxyapatite, barium sulfate, polyurethane powders, composite fillers, hollow silica microspheres, silica microspheres such as, for example, those sold under the names Solesphere H51 or Sunsphere H51 by AGC SI-Tech, and glass or ceramic microcapsules. Use may also be made of particles, which have the shape of portions of hollow spheres, as described in patent applications JP-2003128788 et JP-2000191789.

In particular, if the composition comprises such fillers, they may be present in a content ranging from 0.3% to 10% by weight, in particular from 1% to 7% by weight, relative to the total weight of the composition.

### Optional Common Additives

In the context of the present invention, the composition may also contain at least one adjuvant chosen from those usually used in the cosmetics field, in particular for makeup and/or care compositions for the skin and the lips.

Examples that may be mentioned include dyestuffs that are soluble in the composition, other than the natural dyes (A) described previously, preserving agents, antioxidants, surfactants, moisturizers, fragrances, bactericides, etc.

These adjuvants and the concentrations thereof must be such that they do not modify the property sought for the composition of the invention, and in particular do not impair the colour stability properties. In accordance with a particular embodiment of the invention, the content of optional additive(s) ranges between 2% and 4% by weight relative to the total weight of the composition.

### Process for Preparing the Composition

The composition according to the invention can be produced by the known processes generally used in the field.

For example, it may be produced via the following process:

First, the pigments are milled in a portion of the fatty compounds, more generally in a portion of the oils of the composition, for example using a three-roll mill.

Moreover, the remainder of the fatty compounds, such as the first solid compound(s) (B), the remaining oils, are mixed and brought to a temperature higher than the melting temperature of the solid compounds, in particular to a temperature of about 90 to 100° C.

If the composition comprises an inorganic thickener, this compound is advantageously added during this step of melting the solid fatty compounds.

Once the mixture has been homogenized, the milled pigment mixture can be introduced, with stirring, as can other additional ingredients, such as fillers in particular.

The natural dye(s) are preferably added to the resulting mixture, after homogenization, with stirring, for example by means of a Rayneri type turbine mixer.

If the composition comprises dyestuffs of pearlescent agent type, it is then preferable to add them at the end of the process for preparing the composition.

Finally, the composition is poured into a mould capable of giving it the shape of a stick or wand, or else into a cup. The composition may then be cooled or allowed to cool to ambient temperature to obtain a solid composition.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

In addition, the sum of the amounts of the ingredients of the composition represents 100% by weight of the composition.

The invention is illustrated in greater detail by the examples presented below.

Unless otherwise indicated, the amounts indicated are expressed as mass percentages.

The examples that follow are presented as non-limiting illustrations of the invention.

### EXAMPLES

### Example 1

In the following table, the amount of each compound is given as a weight percentage/weight of composition (the percentages are expressed as weight of starting material unless otherwise indicated).

TABLE 1

| Ingredients (INCI name) | Concentration |
|---|---|
| CAPRYLIC/CAPRIC TRIGLYCERIDE | 10.37 |
| (DUB MCT 7030/MB; Stéarineries Dubois) | |
| CANOLA OIL (Lipex Preact; Aarhuskarlshamn) | 17.84 |
| SQUALANE (Plantasens Olive squalane; Clariant) | 12.49 |
| BIS-BEHENYL/ISOSTEARYL/PHYTOSTERYL DIMER DILINOLEYL | 20 |
| DIMER DILINOLEATE | |
| (Plandool G; Nippon Fine Chemical) | |
| POLYGLYCERYL-4 DIISOSTEARATE/POLYHYDROXYSTEARATE/SEBACATE | 1 |
| (Isolan GPS; Evonik) | |
| COPERNICIA CERIFERA (CARNAUBA) WAX | 1.97 |
| HELIANTHUS ANNUUS (SUNFLOWER) SEED WAX | 8.27 |
| BEESWAX | 3.82 |
| CETYL PALMITATE | 3.94 |
| TITANIUM DIOXIDE | 1.00 |
| IRON OXIDES (and) DISODIUM STEAROYL GLUTAMATE (and) | 0.2 |
| ALUMINIUM HYDROXIDE | |
| IRON OXIDES (and) DISODIUM STEAROYL GLUTAMATE (and) | 0.40 |
| ALUMINIUM HYDROXIDE | |
| CARMINE (Unipure Red LC320; Sensient) | 0.5 |
| BETA-CAROTENE (Natpure Col Red LC 318L; Sensient) | 0.50 |
| ANNATTO (Yellow/Orange - OS - L - HO600480; Naturex) | 0.5 |
| Inverse emulsion of anthocyanin dye according to the | 10.00 |
| example of WO 2017/162599 | |
| Cellulose (Cellulobeads D-10; Daito Kasei Kogyo) | 6.1 |
| Tocopherol | 0.50 |
| Fragrance | 0.70 |

### Preparation of the Composition

1. On the one hand, the solid pigments are milled in some of the oils of the formula in a three-roll mill.
2. On the other hand, the remainder of the oils, the pasty compound, the waxes and the surfactant are brought to a temperature of about 90-95° C., with stirring.
3. The natural dyes and the fillers are then added and the whole combination is mixed by means of a Rayneri type stirring turbine (deflocculator), and finally the fragrance and tocopherol are added.
4. The composition obtained, at 90-95° C., is then poured into appropriate lipstick moulds.
5. The wands are removed from the mould once the composition has solidified, and packaged.

### Evaluation of the Composition

The composition is easily applied to the lips, in a precise, uniform and comfortable deposit.

After application, the colour remains stable on the lips.

Instrumental screening result (measured in accordance with the protocol in description): ΔE_{2000(1:1:1)}=1.1

### Comparative Example 2

The composition below is devoid of dimer dilinoleyl dimer dilinoleate pasty compound and comprises hydroxylated polar oils.

TABLE 2

| Ingredients (INCI name) | Concentration |
|---|---|
| OCTYLDODECANOL | 17.8 |
| DIISOSTEARYL MALATE | 15.6 |
| PETROLATUM | 13.3 |
| POLYBUTENE (INDOPOL H 100; Ineos) | 20.7 |
| BUTYROSPERMUM PARKII (SHEA) BUTTER | 2.5 |
| (Organic deodorized shea butter; Emile Noel company) | |
| HYDROGENATED OLIVE OIL MYRISTYL ESTERS | 4 |
| (Phytowax Olive 14L48; Sophim) | |
| BIS-DIGLYCERYL POLYACYLADIPATE-2 | 6.7 |
| (Softisan 649; Cremer Oleo) | |
| POLYETHYLENE (PERFORMALENE 500-L | 5.3 |
| Polyethylene; Nucera Solutions) | |
| Inverse emulsion of anthocyanin dye according to | 10 |
| the example of WO2017/162599 | |
| TITANIUM DIOXIDE | 1 |
| HDI/TRIMETHYLOL HEXYLLACTONE | 1.8 |
| CROSSPOLYMER (D-400; Toshiki pigments) | |

### Preparation of the Composition

1. On the one hand, the titanium dioxide is milled in some of the oils of the formula in a three-roll mill.
2. On the other hand, the remainder of the oils, the pasty compound, and the waxes are brought to a temperature of about 90-95° C., with stirring.
3. The natural dyes and the filler are then added and the whole combination is mixed by means of a Rayneri type stirring turbine (deflocculator).
4. The composition obtained, at 90-95° C., is then poured into appropriate lipstick moulds.
5. The wands are removed from the mould once the composition has solidified, and packaged.

### Evaluation of the Composition

The composition is easily applied to the lips, in a precise, uniform deposit.

However, after application, the colour changes on the lips.

Instrumental screening result (measured in accordance with the protocol in description): ΔE_{2000(1:1:1)}=3.2

### Comparative Example 3

The composition below comprises hydroxylated oils.

TABLE 3

| Ingredients (INCI name) | Concentration |
|---|---|
| CAPRYLIC/CAPRIC TRIGLYCERIDE | 10.37 |
| (DUB MCT 7030/MB; Stéarineries Dubois) | |
| CANOLA OIL (Lipex Preact; Aarhuskarlshamn) | 31.04 |
| RICINUS COMMUNIS (CASTOR) SEED OIL | 12.50 |
| BIS-BEHENYL/ISOSTEARYL/PHYTOSTERYL | 8.50 |
| DIMER DILINOLEYL DIMER DILINOLEATE | |
| (Plandool G; Nippon Fine Chemical) | |
| POLYGLYCERYL-2 TRIISOSTEARATE | 10.00 |
| COPERNICIA CERIFERA (CARNAUBA) WAX | 1.73 |
| HELIANTHUS ANNUUS (SUNFLOWER) SEED WAX | 7.29 |
| BEESWAX | 2.31 |
| CETYL PALMITATE | 3.47 |
| TITANIUM DIOXIDE | 1.00 |
| IRON OXIDES (and) DISODIUM STEAROYL | 0.40 |
| GLUTAMATE (and) ALUMINIUM HYDROXIDE | |
| BETA-CAROTENE | 0.20 |
| (Natpure Col Red LC 318L; Sensient) | |
| Inverse emulsion of anthocyanin dye according | 10.00 |
| to the example of WO 2017/162599 | |
| TOCOPHEROL | 0.50 |
| FRAGRANCE | 0.70 |

### Preparation of the Composition

1. On the one hand, the solid pigments are milled in some of the oils of the formula in a three-roll mill.
2. On the other hand, the remainder of the oils, the pasty compound, the waxes and the surfactant are brought to a temperature of about 90-95° C., with stirring.
3. The natural dyes and the fillers are then added and the whole combination is mixed by means of a Rayneri type stirring turbine (deflocculator), and finally the fragrance and tocopherol are added.
4. The composition obtained, at 90-95° C., is then poured into appropriate lipstick moulds.
5. The wands are removed from the mould once the composition has solidified, and packaged.

### Evaluation of the Composition

The composition is easily applied to the lips, in a precise, uniform and comfortable deposit.

After application, the colour changes visibly and significantly on the lips.

### Comparative Example 4

The composition below comprises hydroxylated oils.

TABLE 4

| Ingredients (INCI name) | Concentration |
|---|---|
| CAPRYLIC/CAPRIC TRIGLYCERIDE | 23 |
| (DUB MCT 7030/MB; Stéarineries Dubois) | |
| CANOLA OIL | 10 |
| (Lipex Preact; Aarhuskarlshamn) | |
| RICINUS COMMUNIS (CASTOR) SEED OIL | 20 |
| BIS-BEHENYL/ISOSTEARYL/PHYTOSTERYL DIMER | 15 |
| DILINOLEYL DIMER DILINOLEATE | |
| (Plandool G; Nippon Fine Chemical) | |
| POLYGLYCERYL-4 DIISOSTEARATE/POLYHYDROXYSTEARATE/SEBACATE | 1 |
| (Isolan GPS; Evonik) | |
| COPERNICIA CERIFERA (CARNAUBA) WAX | 1.97 |
| HELIANTHUS ANNUUS (SUNFLOWER) SEED WAX | 8.27 |
| BEESWAX | 3.82 |
| CETYL PALMITATE | 3.94 |
| TITANIUM DIOXIDE | 1.00 |
| IRON OXIDES (and) DISODIUM STEAROYL GLUTAMATE | 0.2 |
| (and) ALUMINIUM HYDROXIDE | |
| IRON OXIDES (and) DISODIUM STEAROYL GLUTAMATE | 0.40 |
| (and) ALUMINIUM HYDROXIDE | |
| CARMINE (UNIPURE RED LC320; Sensient) | 0.5 |
| BETA-CAROTENE | 0.50 |
| (Natpure Col Red LC 318L, Sensient) | |
| ANNATTO | 0.5 |
| (YELLOW/ORANGE - OS - L - HO600480; Naturex) | |
| Inverse emulsion of anthocyanin dye according | 10.00 |
| to the example of WO 2017/162599 | |
| Cellulose (Cellulobeads D-10; Daito Kasei Kogyo) | 6 |
| TOCOPHEROL | 0.50 |
| FRAGRANCE | 0.70 |

### Preparation of the Composition

1. On the one hand, the solid pigments are milled in some of the oils of the formula in a three-roll mill.
2. On the other hand, the remainder of the oils, the pasty compound, the waxes and the surfactant are brought to a temperature of about 90-95° C., with stirring.
3. The natural dyes and the fillers are then added and the whole combination is mixed by means of a Rayneri type stirring turbine (deflocculator), and finally the fragrance and tocopherol are added.
4. The composition obtained, at 90-95° C., is then poured into appropriate lipstick moulds.
5. The wands are removed from the mould once the composition has solidified, and packaged.

### Evaluation of the Composition

The composition is easily applied to the lips, in a precise, uniform and comfortable deposit.

After application, the colour changes on the lips significantly.

1. Solid composition for making up and/or caring for keratin materials such as the skin and/or lips, comprising:
(A) at least one natural dyestuff or dyestuff of natural origin chosen from anthocyanins, anthocyanidins, proanthocyanidins, and mixtures thereof;
(B) at least one first compound that is solid at 25° C., chosen from polyesters obtained at least from a mono-unsaturated or poly-unsaturated fatty acid dimer; the fatty acid comprising from 16 to 22 carbon atoms;
(C) at least one volatile or non-volatile first oil, chosen from apolar hydrocarbon oils; silicone oils; liquid polyesters obtained from a mono-unsaturated or poly-unsaturated fatty acid dimer, the fatty acid comprising from 16 to 22 carbon atoms; ester, ether or carbonate hydrocarbon oils, these oils being other than the abovementioned liquid polyesters and being free of free hydroxyl groups; plant oils with the exception of castor oil and of lesquerella oil; and mixtures thereof.

2. Composition according to claim 1, characterized in that the at least one first compound that is solid at 25° C. is chosen from Bis-Behenyl/Isostearyl/Phytosteryl Dimer Dilinoleyl Dimer Dilinoleate, Phytosteryl/Isosteryl/Cetyl/Stearyl/Behenyl Dimer Dilinoleate, Hydrogenated Castor Oil Dimer Dilinoleate, Diglycerin/Dilinoleic Acid/Hydroxystearic Acid Copolymer, and also mixtures thereof.

3. Composition according to claim 1, characterized in that the content of the at least one first compound that is solid at 25° C. is from 10% to 35% by weight relative to the total weight of the composition.

4. Composition according to claim 1, characterized in that the at least one volatile or non-volatile first oil comprises at least one non-volatile oil chosen from:
apolar hydrocarbon oils chosen from liquid paraffin, squalane, isoeicosane, C₁₅₋₁₉ alkane, C₁₈₋₂₁ alkane, C₂₁₋₂₈ alkane, hydrogenated or non-hydrogenated polybutenes, hydrogenated or non-hydrogenated polyisobutenes, hydrogenated or non-hydrogenated polydecenes, and mixtures thereof;
non-phenylated silicone oils chosen from dimethicones, Trimethyl Pentaphenyl Trisiloxane, Tetramethyl Tetraphenyl Trisiloxane, Diphenyl Dimethicones, Trimethylsiloxyphenyl Dimethicones, Phenyltrimethicone, Diphenylsiloxyphenyl Trimethicone, and mixtures thereof;
liquid polyesters obtained from dilinoleic dimer acid, and from at least one C₃-C₄ diol; from an alcohol dimer, the alcohol of which is C₁₆-C₃₂, and mixtures thereof;
hydrocarbon ester oils, other than the abovementioned liquid polyesters, comprising 1 to 4 ester functions, comprising in total between 17 and 80 carbon atoms, which are linear or branched, and saturated, unsaturated or aromatic, free of free hydroxyl groups;
plant oils with the exception of castor oil and of lesquerella oil,
dicaprylyl ether,
dicaprylyl carbonate,
and mixtures thereof.

5. Composition according to claim 1, characterized in that the at least one volatile or non-volatile first oil comprises at least one non-volatile oil chosen from squalane, Dimer dilinoleyl Dimer dilinoleate, Dilinoleic acid/Butanediol copolymer, dilinoleic acid/Propanediol copolymer, capric/caprylic acid triglycerides, oleyl erucate, pentaerythrityl tetraisostearate, olive oil, sunflower oil, apricot oil, sweet almond oil, argan oil, rapeseed oil, canola oil, jojoba oil, sesame oil, and mixtures thereof.

6. Composition according to claim 1, characterized in that the at least one volatile or non-volatile first oil is chosen from apolar volatile hydrocarbon oils, linear, branched or cyclic volatile silicone oils; and mixtures thereof.

7. Composition according to claim 1, characterized in that the content of the at least one volatile or non-volatile first oil is between 40% and 60% by weight relative to the total weight of the composition.

8. Composition according to claim 1, characterized in that the content of volatile oil(s) in the composition is less than or equal to 15% by weight relative to the total weight of the composition.

9. Composition according to claim 1, characterized in that it further comprises at least one lipophilic thickener chosen from silicas, dextrin esters, and mixtures thereof.

10. Composition according to claim 9, characterized in that the content of lipophilic thickener(s) is from 0.1% to 10% by weight relative to the total weight of the composition.

11. Composition according to claim 1, characterized in that it further comprises at least one lipophilic thickener chosen from natural or synthetic clays in a content of less than or equal to 2% by weight, relative to the total weight of the composition.

12. Composition according to claim 1, characterized in that it further comprises water in a content of less than or equal to 10% by weight relative to the total weight of the composition.

13. Composition according to claim 1, characterized in that it further comprises at least one hydroxylated solvent chosen from monoalcohols containing from 1 to 5 carbon atoms, C₂-C₈ polyols that are liquid at ambient temperature and atmospheric pressure, comprising from 2 to 4 free hydroxyl groups, and mixtures thereof, in a content of less than or equal to 10% by weight relative to the total weight of the composition, and optionally further comprises water, with a combined amount of water and of hydroxylated solvent(s) being less than or equal to 10% by weight, relative to the total weight of the composition.

14. Composition according to claim 1, characterized in that it further comprises at least one solid fatty compound (D) chosen from:
waxes chosen from apolar hydrocarbon waxes, hydrocarbon ester waxes chosen from fatty acid monoesters, dicarboxylic acid diesters, total esters of glycerol and of a C₁₆-C₃₀ carboxylic acid, these waxes not comprising a free hydroxyl group; waxes of animal or plant origin, with the exception of carnauba wax; waxes obtained by hydrogenation of animal or plant oils, with the exception of hydrogenated castor oil, which has been optionally esterified; hydrogenated lesquerella oil; and mixtures thereof;
pasty compounds chosen from petroleum jelly; lanolins and derivatives; butters of plant origin; Dipentaerythrityl tetrahydroxystearate/tetraisostearate; triglycerides of saturated fatty acids or hydrogenated fatty acids; wholly or partially hydrogenated plant oils, optionally partially hydrogenated, with the exception of hydrogenated castor oil; and mixtures thereof.

15. Composition according to claim 1, characterized in that the composition further comprises at least one liquid or solid additional compound, chosen from oils, waxes, pasty compounds, and mixtures thereof other than at least one first compound that is solid at 25° C. (B) and the at least one volatile or non-volatile first oil (C), chosen from C₈-C₆₀ fatty alcohols; hydroxylated esters; hydrogenated or non-hydrogenated castor oil, which has optionally been esterified; hydrogenated or non-hydrogenated lesquerella oil; glyceryl trihydroxystearate; carnauba wax; monooxyalkylenated or polyoxyalkylenated hydrocarbon or silicone compounds, the alkyl group(s) of which is/are of C₂-C₃, mono-glycerolated or poly-glycerolated hydrocarbon or silicone compounds; bis-diglyceryl polyacyladipate-2; and mixtures thereof.

16. Composition according to claim 15, characterized in that the content of additional liquid compound(s) is less than a content of the at least one volatile or non-volatile first oil (C).

17. Composition according to claim 15, characterized in that a content of additional solid compound(s) other than the at least one first compound that is solid at 25° C. (B), if present, is less than a content of the at least one first compound that is solid at 25° C. (B).

18. Composition according to claim 13, characterized in that a total content of water and/or of hydroxylated solvent(s) and/or of glyceryl trihydroxystearate, if present, is less than or equal to 10% by weight, relative to the total weight of the composition.

19. Composition according to claim 1, characterized in that the at least one natural dyestuff or dyestuff of natural origin come(s) from at least one plant chosen from chokeberry, blueberry, cranberry, purple or black carrot, blackcurrant, cherry, red cabbage, poppy, strawberry, geranium, pomegranate, hibiscus, purple corn, bilberry, purple sweet potato, red radish, black grape, sorghum, elderberry, and mixtures thereof.

20. Composition according to claim 1, characterized in that the content of the least one natural dyestuff or dyestuff of natural origin, expressed as dyestuff active material, is between 0.5% and 20% by weight relative to the total weight of the composition.

21. Composition according to claim 1, characterized in that it further comprises at least one additional dyestuff, other than the at least one natural dyestuff or dyestuff of natural origin; the content of additional dyestuff(s) being such that the composition exhibits a variation in coloring under the conditions below:
Protocol:
The two samples below are prepared:
Sample 1
2.5 g of composition according to the invention
0.5 g octyldodecanol
1 g of pH 10 buffer solution
The composition is crushed with a spatula and mixed with the rest of the ingredients for one minute at 3000 rpm (for example by means of a speed mixer).
Sample 2
2.5 g of composition according to the invention
0.5 g octyldodecanol
1 g of solution of water+citric acid at pH 3
The composition is crushed with a spatula and mixed with the rest of the ingredients for one minute at 3000 rpm (for example by means of a speed mixer).
Measurement
One hour after the preparation of each sample, an appropriate amount of mixture is spread as a deposit of uniform thickness using a Multiple Clearance Square Applicator 5363, sold by Byk-Gardner (thickness 2 mils, i.e. 51 μm) onto the white side of a contrast card (unvarnished Byk card, ref. 2831);
The color of the deposit of Sample 1 on the one hand, and of Sample 2 on the other hand, is measured after 24 hours by means of a CM600d colorimeter from Konica Minolta in the CIELab 76 system (illuminant D65, SCI, 10° Observer) at three different locations away from the edges of the deposit, and the L*, a and b* coordinates of the color are obtained in the CIELab 76 space;
The mean of each L*, a*, b* coordinate for each sample is then calculated;
The variation in coloration, evaluated from the calculation of the mean ΔE₂₀₀₀ according to the ISO 11664-6:2014 standard (specification of the method of calculation of the color differences according to the formula CIEDE2000), is greater than 2 in absolute value.

22. Process for making up and/or caring for a keratin material comprising applying the composition according to claim 1 to the keratin material in an amount sufficient to make up and/or care for the keratin material.
